(19) United States
(12) Patent Application Publication
(10) Pub. No.: US 2017/0332913 A1
(43) Pub. Date: Nov. 23, 2017

(54) OBJECT INFORMATION ACQUIRING APPARATUS

(71) Applicant: CANON KABUSHIKI KAISHA, Tokyo (JP)

(72) Inventor: Yoshiko Nakamura, Kusatsu-shi (JP)

(21) Appl. No.: 15/520,899

(22) PCT Filed: Nov. 2, 2015
(86) PCT No.: PCT/JP2015/005499

§ 371 Date: Apr. 21, 2017

(30) Foreign Application Priority Data
Nov. 7, 2014 (JP) 2014-226951

(51) Int. Cl. A61B 5/00

(52) U.S. Cl. CPC A61B 5/0095; A61B 5/6844; A61B 2562/046

(57) ABSTRACT

An object information acquiring apparatus comprises a receiving unit including an acoustic wave detection elements; a setting unit configured to set to the object an area of interest for forming characteristics information on the object; a position control unit configured to move the receiving unit to predetermined reception positions; a recording unit storing information on the time taken for the acoustic waves generated from the area of interest to reach the acoustic wave detection elements and information on the predetermined reception positions after associating these two pieces of information; and an acquiring unit configured to acquire the characteristics information on the object using the electrical signals corresponding to the acoustic waves propagated from the area of interest and also using the information on the time.

### TECHNICAL FIELD

The present invention relates to an object information acquiring apparatus.

### BACKGROUND ART

A photoacoustic tomography (PAT) apparatus used in medical diagnosis, for example, is proposed as an apparatus for reconstructing the inside of an examination object (object) using ultrasound waves (acoustic waves).

Photoacoustic tomography involves irradiating an object with a pulsed laser beam, receiving photoacoustic waves generated when a tissue inside the object absorbs the energy of the irradiation beam using an acoustic wave detection element, and reconstructing information on an optical characteristic value of the inside of the object. Here, photoacoustic tomography reconstructs images assuming that the acoustic velocity in a living body (object), used when reconstructing images is constant. However, the actual acoustic velocity in the living body is not constant. Thus, if the acoustic velocity used for reconstruction is set to be constant, the actual acoustic velocity in the living body is not identical to the set value. As a result, when images are reconstructed based on the set value, the contrast and the definition of the reconstructed image decrease.

In this respect, a method of reconstructing images based on acoustic waves is proposed. According to this method, the acoustic velocity inside an object is calculated based on acoustic waves from a designated area and image reconstruction is performed using the acoustic velocity (Patent Literature 1).

### CITATION LIST

### Patent Literature

[PTL 1] Japanese Patent Application Publication No. 2011-120765

### SUMMARY OF INVENTION

### Technical Problem

According to the method disclosed in Patent Literature 1, an acoustic velocity in a designated area is calculated by calculating an acoustic velocity at which the phases of photoacoustic signals before reconstruction are aligned, and images with higher quality are obtained by performing reconstruction based on the acoustic velocity. However, an acoustic velocity table is different from path to path along which acoustic waves propagate through a measurement object, and a process of calculating an optimal acoustic velocity incurs a considerable amount of time. Thus, when images are generated by performing reconstruction using an optimal acoustic velocity, it is necessary to calculate the optimal acoustic velocity every reconstruction and a large amount of time is required for the calculation.

In view of the above problems, an object of the present invention is to provide an object information acquiring apparatus capable of reducing the time to acquire characteristics information of an object with a simple structure and improving the accuracy of the characteristics information on the object.

### Solution to Problem

In order to attain the object, the present invention includes the following structure. That is, an object information acquiring apparatus includes: a receiving unit including a plurality of acoustic wave detection elements configured to receive acoustic waves propagating through an object and output electrical signals; a setting unit configured to set to the object an area of interest for forming characteristics information on the object; a position control unit configured to move the receiving unit to a plurality of predetermined reception positions; a recording unit storing, with respect to each of the plurality of predetermined reception positions, information on the time taken for the acoustic waves generated from the area of interest to reach the plurality of acoustic wave detection elements and information on the predetermined reception positions after associating these two pieces of information; and an acquiring unit configured to acquire the characteristics information on the object in the area of interest with respect to each of the plurality of predetermined reception positions, using the electrical signals output when the plurality of acoustic wave detection elements have received the acoustic waves propagated from the area of interest and also using the information on the time read from the recording unit.

The present invention in its another aspect provides an object information acquiring method comprising a step of moving a plurality of acoustic wave detection elements configured to receive acoustic waves propagating through an object and to output electrical signals, to a plurality of predetermined reception positions; a step of receiving the acoustic waves using the plurality of acoustic wave detection elements and acquiring electrical signals; a step of setting an area of interest to the object for forming characteristics information on the object; a step of acquiring information on the time taken for the acoustic waves generated from the area of interest to reach the plurality of acoustic wave detection elements and acquiring corresponding information on the predetermined reception positions, with respect to each of the plurality of predetermined reception positions; and a step of acquiring the characteristics information on the object in the area of interest with respect to each of the plurality of predetermined reception positions, using the electrical signals output when the plurality of acoustic wave detection elements have received the acoustic waves propagated from the area of interest and also using the information on the time.

### Advantageous Effects of Invention

As described above, according to the present invention, it is possible to provide an object information acquiring apparatus capable of reducing the time to acquire characteristics information on an object with a simple structure and improving the accuracy of the characteristics information on the object.

Further features of the present invention will become apparent from the following description of exemplary embodiments with reference to the attached drawings.

### BRIEF DESCRIPTION OF DRAWINGS

FIG. 1 is a block diagram illustrating Example 1 of an object information acquiring apparatus according to the present invention.

FIG. 2 is a schematic diagram illustrating the relation between an area of interest and a receiving unit of Example 1.

FIG. 3 is a diagram illustrating the results shown in a delay period table according to Embodiment 1.

FIGS. 4A and 4B are schematic diagrams illustrating Example 2 of an object information acquiring apparatus according to the present invention.

FIG. 5 is a diagram illustrating a delay period table according to Example 2.

FIG. 6 is a diagram illustrating an acoustic velocity V in each area of interest obtained by dividing an object according to Example 2.

FIGS. 7A to 7C are schematic diagrams illustrating scanning implemented according to Example 4 of the object information acquiring apparatus of the present invention.

### DESCRIPTION OF EMBODIMENTS

Hereinafter, embodiments of the present invention will be described with reference to the drawings. As a general rule, the same constituent elements will be denoted by the same reference numerals, and the description thereof will be omitted. Detailed calculation formula, calculation procedures, and the like described below are to be appropriately changed according to the configuration and various conditions of an apparatus to which the present invention is applied, and the scope of the present invention is not limited to those described below.

An object information acquiring apparatus (photoacoustic tomography or the like) of the present invention includes an apparatus which uses a photoacoustic effect to receive acoustic waves generated inside an object by irradiating the object with light (electromagnetic waves) such as near-infrared light to acquire object information as image data. In the case of an apparatus which uses a photoacoustic effect, the acquired object information indicates a generation source distribution of acoustic waves generated by light irradiation, an initial acoustic pressure distribution inside the object, an optical energy absorption density distribution and an absorption coefficient distribution derived from the initial acoustic pressure distribution, or a concentration distribution of a substance that constitutes a tissue. Examples of the substance concentration distribution include an oxygen saturation distribution, a total hemoglobin concentration distribution, and an oxygenated or reduced hemoglobin concentration distribution.

Moreover, the characteristics information on the interior of the object at a plurality of positions thereof may be acquired as a 2-dimensional or 3-dimensional characteristics distribution. The characteristics distribution is generated as image data indicating the characteristics information on the interior of an object. The acoustic wave referred in the present invention is typically an ultrasound wave and includes those referred to as a sound wave and an acoustic wave. The acoustic wave generated by the photoacoustic effect is referred to as a photoacoustic wave or an optical ultrasonic wave. An acoustic wave detection element (probe) receives acoustic waves generated inside or reflected from an object.

The object information acquiring apparatus can be configured of various constituent elements, and in combination of the constituent elements, and also in combination of process operations. The drawings are presented to illustrate preferred embodiments and do not restrict the present invention.

In the following description, a “reception position” indicates the position at which a receiving unit 105 receives photoacoustic waves and an “element position” indicates the coordinate (position) of each acoustic wave detection element 106 when photoacoustic waves are received.

### Example 1

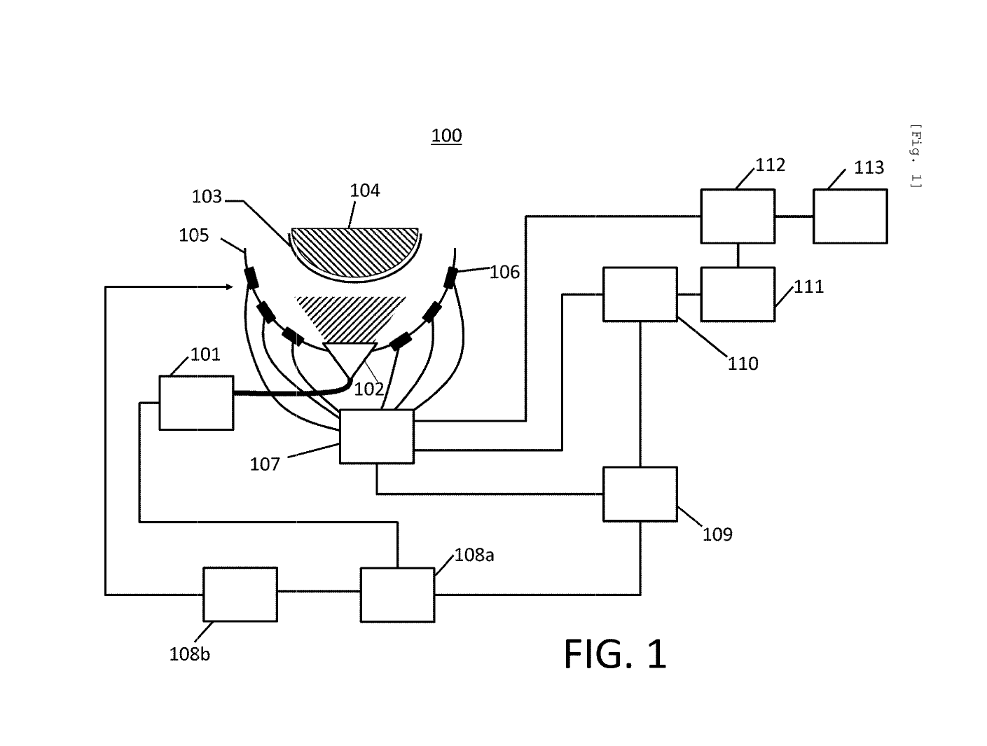

FIG. 1 is a block diagram illustrating Example 1 of an object information acquiring apparatus of the present invention. An object information acquiring apparatus 100 (hereinafter referred to simply as an “apparatus 100”) includes a light source 101, an irradiating unit 102, and an acoustic matching material 103. The apparatus 100 further includes a receiving unit 105, an acoustic wave detection element 106 (hereinafter referred to simply as an “element 106”), a signal processing unit 107, a drive control unit 108a (corresponding to a position control unit), and a driving unit 108b (corresponding to the position control unit). The apparatus 100 further includes a selecting unit 109 (corresponding to a setting unit), a calculating unit 110, a recording unit 111, a reconstructing unit 112 (corresponding to an acquiring unit), and a display unit 113.

The apparatus 100 generates a pulsed light beam from the light source 101, guides the pulsed light beam to the irradiating unit 102, and irradiates an object 104 with the pulsed light beam with the aid of the irradiating unit 102. With the pulsed light beam radiated to the object 104, a light absorber (that is, hemoglobin and the like in the blood) in the object 104 generates photoacoustic waves.

The irradiating unit 102 irradiates the object 104 with the pulsed light beam from the light source 101 when the receiving unit 105 is positioned at a predetermined reception position.

The light source 101 may generate a pulsed light beam having a width of nanoseconds. The light generated by a light source having a wavelength of 700 nm or smaller cannot sufficiently reach a deep portion inside the object 104 since a large portion thereof is absorbed by hemoglobin, collagens, and the like. Thus, it is preferable to use a wavelength of 700 nm or larger. The wavelength used in the present example may be changed according to a constituent substance (a cancer, a blood vessel, fat, or the like) included in the object 104. This is because constituent substances have different optical absorption spectra. Although a laser is preferably used as the light source 101 in order to obtain a large output, a light-emitting diode or the like may be used instead of the laser. Alternatively, a flash lamp or the like may be used. Various lasers such as a solid laser, a gas laser, a dye laser, or a semiconductor laser can be used as the laser. The timing, waveform, intensity, and the like of light irradiation are controlled by the drive control unit 108a. Moreover, in order to guide light from the light source 101 to the object 104, an optical member such as a mirror that reflects light, a lens that focuses or diffuses light or changes the shape thereof, a prism that spreads, refracts, or reflects light, an optical fiber that propagates light, or a diffuser may be used.

The element 106 receives photoacoustic waves through the acoustic matching material 103. Since the receiving unit 105 has a plurality of elements 106 arranged along an approximately hemispherical shape of the receiving unit, the receiving unit 105 can acquire photoacoustic waves from a light absorber at a plurality of positions simultaneously. In this way, the receiving unit 105 can reduce the time taken to receive photoacoustic waves and suppress the influence such as vibration of the object 104. The element 106 receives photoacoustic waves propagating from the object 104 and converts the photoacoustic waves to electrical signals (hereinafter referred to appropriately as “reception signals” or “reception results”) according to a piezoelectric effect. A piezoelectric element (a piezo element or the like) which is a conversion element which uses a piezoelectric phenomenon, a conversion element which uses resonance of light, a conversion element which uses a change in capacitance, and other element can be used as the element 106. However, the element 106 is not limited thereto, but an arbitrary element may be used as long as the element can receive photoacoustic waves from the object 104 and convert the photoacoustic waves into electrical signals.

The receiving unit 105 is formed in an approximately hemispherical shape (cup shape), and the plurality of elements 106 are arranged in a spiral form along the approximately hemispherical shape. The receiving unit 105 is driven to rotate (revolve) about a rotation axis at the center of the opening surface and vertical to a cup-shaped opening surface or to translate intermittently or continuously. In the present example, although a scanning operation is performed using translational movement only without using revolution, the present invention is not limited thereto, but the scanning operation may be performed using revolution. The receiving unit 105 receives photoacoustic waves at a certain reception position with this translational movement and moves to a next reception position for receiving subsequent photoacoustic waves. When the receiving unit 105 is positioned at a certain reception position, photoacoustic waves generated by the light irradiation of the irradiating unit 102 are received by all elements 106. A signal memory included in the signal processing unit 107 stores the reception result (electrical signal) of each element 106 in association with the element position of each element. This process is performed at all reception positions. That is, all reception signals of all elements 106 at each reception position are stored in the signal memory in association with the element position of each element 106.

The drive control unit 108a outputs a control signal for performing the translational driving of the receiving unit 105 to the driving unit 108b. Moreover, the drive control unit 108a outputs an emission timing control signal for controlling the emission timing of the light source 101 to the light source 101. This emission timing is used to allow the light source 101 to emit light when the receiving unit 105 is at the position for receiving photoacoustic waves.

The driving unit 108b receives the control signal from the drive control unit 108a to allow the receiving unit 105 to translate to the reception position for photoacoustic waves based on the control signal. The driving unit 108b moves the receiving unit 105 to an ending position to finish the scanning operation after the receiving unit 105 receives photoacoustic waves at the last reception position. The driving unit 108b may continuously move the receiving unit 105 at the same velocity along a moving path of the receiving unit 105 that passes through the respective reception positions and may sequentially move the receiving unit 105 to the respective reception positions in a stepwise manner.

The driving unit 108b may move the irradiating unit 102 so that a pulsed light beam moves. In the present example, the irradiating unit 102 moves with the movement of the receiving unit 105. The driving unit 108b may include a motor such as a step motor that generates driving force, a driving mechanism that transmits driving force, and a position sensor that detects position information of the receiving unit 105. A lead screw mechanism, a link mechanism, a gear mechanism, a hydraulic mechanism, or the like may be used as the driving mechanism. Moreover, an encoder, a potentiometer such as a variable resistor, or the like may be used as the position sensor. The driving unit 108b can change the relative position between the object 104 and the receiving unit 105 one, two, or three-dimensionally. The driving unit 108b may change the relative position between the object 104 and the irradiating unit 102 or the receiving unit 105. That is, the driving unit 108b may move at least one of the irradiating unit 102, the receiving unit 105, and the object 104. Moreover, the driving unit 108b may continuously move the relative position and may move the relative position in a step-and-repeat manner.

The element 106 receives photoacoustic waves generated based on radiation of pulsed light beams and outputs electrical signals to the signal processing unit 107. In the receiving unit 105, the elements 106 are arranged along the hemispherical surface of the receiving unit 105. By doing so, the receiving unit 105 can allow a direction in which the reception sensitivity of the respective elements 106 is highest to be focused on a predetermined area so that a high-resolution area which is the predetermined area is formed in the object 104.

The reconstructing unit 112 images an area of interest described later using data stored in the recording unit 111 to acquire object information.

The signal processing unit 107 acquires electrical signals which are the reception results of the elements 106. The signal processing unit 107 includes an A/D converter that converts analog electrical signals from the elements 106 into digital electrical signals, an amplifier such as an operational amplifier that amplifies the digital signals which are the conversion results, and a signal memory that stores signals obtained by performing A/D conversion and amplification. The A/D conversion and the signal amplification may be performed in an arbitrary order. That is, the signal processing unit 107 receives photoacoustic waves generated by radiating light at respective reception positions, performs A/D conversion and amplification on the electrical signals transmitted from the respective elements 106 to obtain signals, and then, stores the obtained signals in the signal memory at the element positions of the respective elements 106. That is, the reception signals of the photoacoustic waves acquired by the respective elements 106 at the respective reception positions are sequentially recorded in the signal memory according to scanning of the receiving unit 105 in the translation direction. The data recorded sequentially in this manner is used in the processing in the reconstructing unit 112 and the recording unit 111 on the subsequent stage.

The signal processing unit 107 may perform a process of filtering the electrical signals input from the elements 106 using a band-pass filter. For example, high-frequency noise included in the electrical signals from the elements 106 may be reduced by removing high-frequency noise components using a low-pass filter. Other means for transmitting the photoacoustic wave reception results may be used instead of the electrical signals. For example, signals may be transmitted using radio communication and may be transmitted using a photocoupler.

The drive control unit 108a outputs the position information corresponding to the time at which the receiving unit 105 receives photoacoustic waves to the selecting unit 109. The selecting unit 109 acquires the position information and acquires straight paths from respective elements 106 to respective areas of interest at the reception position. The selecting unit 109 selects at least one element 106 having high reception sensitivity in each area of interest among all elements 106 based on the respective straight paths in order to calculate an optimal acoustic velocity in the respective areas of interest. By doing so, images can be reconstructed using only signals received with higher sensitivity from the acoustic waves that have been generated in respective areas of interest. As a result, the accuracy of the acquired optimal acoustic velocity in the respective areas of interest can be improved.

First, the selecting unit 109 sets an area of interest in a measurement area of the object 104. Here, the measurement area is set for the object 104 and is an area that includes the object 104 and that is to be finally imaged. That is, the measurement area is an area in which the plurality of elements 106 can receive the generated photoacoustic waves. In this example, the area of interest is a plurality of rectangular solid areas set so as to cover an entire measurement area and is an area in which a delay period is set in each area of interest and each element position, and in which images are reconstructed. The size of the area of interest needs to be equal to or larger than one voxel of a reconstructed image. The delay period is a parameter calculated by an equation (Delay period (T)=Distance (L)/Optimal Acoustic Velocity (S)) from an optimal acoustic velocity S allocated to an area of interest and the distance L to the area of interest from the element 106 that output an electrical signal used when calculating the optimal acoustic velocity of the area of interest. When the delay period T is defined in this manner, the delay period T corresponds to the time taken for an acoustic wave generated in an area of interest to reach the selected element 106.

Subsequently, the selecting unit 109 selects data received at an element position optimal for imaging each area of interest from the data received at all element positions, stored in the signal memory. In order to calculate the optimal acoustic velocity, various acoustic velocity values are allocated to the reception data and an image of the area of interest is reconstructed for each acoustic velocity value. Moreover, an acoustic velocity value at which best contrast or the like is obtained is set as the optimal acoustic velocity. The higher the accuracy of the reception data, the better the accuracy of the reconstructed image, and thus, the better the optimal acoustic velocity calculated from the reception data. The reception data at the element position optimal for imaging the area of interest is the following data, for example. That is, the reception data is the data obtained when an element having directivity capable of ideally receiving acoustic waves propagating from a certain area of interest receives the acoustic waves. That is, the reception data is digital data which is obtained when the element receives the acoustic waves, outputs electrical signals, converts the electrical signals into digital signals, which are stored in the signal memory, and which is obtained by performing the storing process in all areas of interest.

The selecting unit 109 selects optimal reception data used for the calculating unit 110 to calculate the optimal acoustic velocity from all items of reception data stored in the signal memory. This selection is performed based on a straight path from an area of interest as an optimal acoustic velocity calculation target to each element position. Specifically, straight paths from an area of interest as an optimal acoustic velocity calculation target to all element positions are acquired, and reception data received by the element 106 in which the angle between the straight line and the direction in which the reception sensitivity of each element 106 is highest is within 40° is selected. The angle is preferably determined according to the directivity of an element and can be appropriately changed.

The selecting unit 109 reads only optimal reception data corresponding to the area of interest as an optimal acoustic velocity calculation target from the signal memory and outputs a selection signal which is a signal for inputting to the calculating unit 110 to the signal processing unit 107. The signal processing unit 107 reads the selected optimal reception data from the signal memory based on the selection signal and inputs the optimal reception data to the calculating unit 110. The calculating unit 110 calculates the optimal acoustic velocity by reconstructing the image of the area of interest as an optimal acoustic velocity calculation target using the optimal reception data input from the signal processing unit 107. The calculating unit 110 calculates the delay period corresponding to the area of interest by dividing the distance from the element position of the element that has received the optimal reception data to the calculation target area of interest by the calculated optimal acoustic velocity.

The recording unit 111 stores the delay period calculated by the calculating unit 110 together with the corresponding area of interest. This process is performed for all areas of interest. Finally, the recording unit 111 stores a table in which the respective areas of interest and the respective delay periods are associated with each other.

The reception data at element positions that can be considered to be the same when seen from a certain area of interest may be averaged when using the same for reconstructing the image of the area of interest. The element positions that can be considered to be the same can be defined as the elements 106 at element positions within a predetermined solid angle range from the area of interest. The solid angle is determined so that an angle between the direction of the element 106 at an optional element position and the straight line from the element 106 to the area of interest is within 3°. However, the present invention is not limited thereto, but the value of the solid angle and a method of selecting the elements 106 that can be considered to be the same may be appropriately changed according to various apparatus configurations. Moreover, a group of element positions at which images are reconstructed using the same acoustic velocity for an area of interest is referred to as an element position group. The element position group is also a group of element positions at which the solid angles fall within a certain solid angle for the area of interest. The solid angle and the method of defining the element position group can be changed according to an apparatus configuration such as the type of an object or the directivity of an element.

When a plurality of light absorbers are adjacent within a measurement area, the plurality of adjacent light absorbers are reconstructed as one light absorber by the calculating unit 110 depending on a method of setting the acoustic velocity reconstructed. Even when photoacoustic measurement of an object is performed using the acoustic velocity acquired through such image reconstruction, the reconstructed image which is the measurement result does not indicate a true light absorber image. That is, in this case, an acoustic velocity that is inappropriate as the optimal acoustic velocity may be set as the optimal acoustic velocity of an area of interest. In order to prevent such a problem, it is important to set the size of an area of interest. When the size of an area of interest is equal to or smaller than the definition or the resolution corresponding to one pixel of the apparatus 100, it may be not possible to calculate the optimal acoustic velocity ideally. Moreover, when the size of an area of interest is equal to or smaller than the definition or the resolution, the number of areas of interest within a measurement range increases and the time to calculate the optimal acoustic velocity also increases. On the other hand, when the area of interest is too large as compared to the definition or the resolution, the acoustic velocity within one area of interest becomes non-uniform and the image characteristics within the reconstructed image becomes uneven. Thus, the accuracy of the reconstructed image may deteriorate and it may be not possible to calculate a desired optimal acoustic velocity. Thus, in the present example, the size of an area of interest is set to a size of 40 mm×40 mm×40 mm which is a range in which the definition deteriorates by 10%. However, the present invention is not limited thereto, but the optimal size of the area of interest may be appropriately set according to the configuration of the apparatus 100, the non-uniformity of the acoustic velocities in the respective paths from the area of interest and the elements 106, or an allowable calculation period.

The calculating unit 110 calculates the optimal acoustic velocity in each area of interest. Further, the calculating unit 110 calculates a delay period in each area of interest by dividing the distance from an element that has received acoustic waves serving as the base of calculation of the optimal acoustic velocity to an area of interest in which the optimal acoustic velocity is calculated by the optimal acoustic velocity. The optimal acoustic velocity in the area of interest mentioned herein is an optimal acoustic velocity in an area of interest corresponding to each of the element positions selected by the above-described method. The following optimal acoustic velocity calculation method may be used, for example. That is, the optimal acoustic velocity is calculated based on the intensity of a reconstructed image of the area of interest. The calculating unit 110 reconstructs images without using the data recorded in the recording unit 111. On the other hand, the function of the reconstructing unit 112 is different from the function of the calculating unit 110 in that the reconstructing unit 112 reconstructs images using the data recorded in the recording unit 111. That is, the calculating unit 110 reconstructs images at a stage in which the optimal acoustic velocity is calculated. In contrast, the reconstructing unit 112 reconstructs images at a stage in which the optimal acoustic velocity is calculated and the delay period is calculated from the optimal acoustic velocity and is recorded in the recording unit 111. That is, the function of the reconstructing unit 112 is different from the function of the calculating unit 110 in that the reconstructing unit 112 performs image reconstruction for acquiring the characteristics information on the object using the recorded delay period at a stage later than the stage of calculating the optimal acoustic velocity. That is, the calculating unit 110 reads optimal reception data which is reception data ideally used for calculating the optimal acoustic velocity corresponding to the area of interest as an optimal acoustic velocity calculation target from the signal memory. Moreover, the calculating unit 110 sets various assumptive optimal acoustic velocity values and reconstructs the image of the area of interest using the optimal reception data with respect to various assumptive acoustic velocity values. That is, a number of images corresponding to the number of assumptive acoustic velocities are reconstructed. The delay period obtained by dividing the distance by the optimal acoustic velocity which is the acoustic velocity used for image reconstruction and which satisfies a predetermined condition among the respective reconstructed images is stored in the recording unit 111 in association with the area of interest.

According to the predetermined condition, the contrast values, the brightness values, or the edge sharpness values of the respective reconstructed images are compared and an assumptive acoustic velocity used when reconstructing an image having the highest intensity is used as the optimal acoustic velocity. In the present example, universal back-projection used in the photoacoustic tomography technique is used as the image reconstruction method. However, the present invention is not limited thereto, but Fourier-domain back-projection, a synthetic aperture method, a time-reversal method which is time-domain back-projection, or the like may be used.

For example, time-domain back-projection (reconstruction) which is a general image reconstruction method is performed as follows. That is, first, a case in which acoustic waves are generated at a position (in this example, an area of interest) of each element 106, corresponding to a black pixel in a reconstructed image will be considered. In this case, the reconstructing unit 112 reads the time ti (delay period: ti−t0 where the generation time t0 is 0) taken for the acoustic waves to reach an i-th element 106 after the acoustic waves are generated from the recording unit 111. Moreover, the intensity S(i,ti) of the reception signal at the time ti is acquired for each element 106, and the intensities are added to generate the pixel of the reconstructed image. Similarly, the pixels are generated at a plurality of positions (in this example, a plurality of areas of interest) to generate the reconstructed image. The intensity S(i,ti) indicates the intensity of a reception signal S(i,t) at the time ti. When the delay period is stored in advance in the recording unit 111, it is possible to eliminate a process of calculating the delay period using an acoustic velocity value after calculating the acoustic velocity value in a preceding stage.

The calculating unit 110 can set an assumptive acoustic velocity in a plurality of substances when performing image reconstruction in order to calculate the optimal acoustic velocity in a preceding stage of calculating the delay period. For example, when water which is an acoustic linker is filled outside the object 104, two acoustic velocities of the assumptive acoustic velocity in the water and the assumptive acoustic velocity of the object 104 may be set. In this case, the calculating unit 110 calculates the optimal acoustic velocities in the water and the object 104 with respect to all areas of interest. However, the present invention is not limited thereto, but the optimal acoustic velocity in water may be calculated from a water temperature and a salinity concentration. The optimal acoustic velocity in the water calculated in this manner may be used in image reconstruction. By doing so, it is possible to reduce the time to calculate the optimal acoustic velocity in the water. In addition to this, if various acoustic matching materials 103 are present, the assumptive acoustic velocities thereof may be set.

The acoustic matching material 103 is a gel filled between a cup and the object 104, the cup for holding the object 104, or the like, for example. That is, if objects in which the acoustic velocities are different are present, the assumptive acoustic velocities thereof may be appropriately additionally set. When the acoustic velocity in the acoustic matching material 103 or the like is known, the known acoustic velocity may be used as the optimal acoustic velocity as it is. Moreover, by acquiring the delay period from the optimal acoustic velocity, storing the delay period in the recording unit 111, and using the delay period to perform image reconstruction during photoacoustic measurement of the object, it is possible to reduce the time to acquire the delay period to be stored in the recording unit 111. Similarly, when the delay period in the acoustic matching material 103 or the like is known, the known delay period may be stored in the recording unit 111 as it is. Moreover, the calculating unit 110 may determine the delay period in each area of interest by determining the optimal acoustic velocity in each area of interest by combining one or a plurality of criteria. When a plurality of criteria are employed, since the difference between the actual acoustic velocity and the optimal acoustic velocity calculated by the calculating unit 110 decreases, the difference between the actual delay period and the delay period calculated from the optimal acoustic velocity decreases. Further, the calculating unit 110 may prepare a plurality of criteria used for determining the optimal acoustic velocity and select the criterion for each area of interest. For example, the calculating unit 110 calculates the optimal acoustic velocity by selecting the contrast and the edge sharpness as the used criteria for an area of interest that is known to have poor definition and acquires the delay period from the optimal acoustic velocity. By doing so, the accuracy of the delay period stored in the recording unit 111 can be improved.

The recording unit 111 records the delay periods acquired in this manner in association with each area of interest and the element position or the element position group. The recording unit 111 records the delay period whenever the delay period in one area of interest is calculated or after the delay periods are calculated for all areas of interest.

When it is determined that a light absorber is not present in an area of interest, the calculating unit 110 may record a predetermined delay period in the delay period table of the recording unit 111 and may record empty data in the recording unit 111. In this case, the reconstructing unit 112 performs the following process when reconstructing images of an area of interest corresponding to the empty data using the data in the recording unit 111. That is, the reconstructing unit 112 performs image reconstruction using an average value of the reception data acquired at element positions at which the straight paths which are expected paths from the element position corresponding to the area of interest to the area of interest can be considered to be the same. The present invention is not limited thereto, but various paths other than the straight path can be used as the expected path. For example, a propagation path in which acoustic waves are attenuated smallest among the propagation paths of acoustic waves from an area of interest to an element can be used. However, the present invention is not limited thereto, but the average value of all delay periods recorded in the recording unit 111 may be used in image reconstruction of an area of interest in which it is determined that the light absorber is not present. The calculating unit 110 may determine that a light absorber is not present in an area of interest according to the following method. That is, the brightness, contrast, definition, or edge sharpness of an image is compared with a predetermined reference value, and when a comparison result (for example, a difference value) does not fall within a predetermined difference range, it is determined that the light absorber is not present in the area of interest. In this case, the predetermined reference value may be a brightness value of a reconstructed image when a light absorber is present in an area of interest, an average value of the brightness values of the reconstructed images of a plurality of areas of interest in which the light absorber is present, or a smallest value of the brightness values of the reconstructed images of a plurality of areas of interest in which the light absorber is present, for example.

The recording unit 111 may be arbitrary means such as a hard disk, an optical disc, or a semiconductor recording medium which can record data. The type of the recording unit 111 is not particularly limited and may be a portable recording medium, a non-portable recording medium, or a cloud recording medium. Moreover, the recording unit 111 may be a nonvolatile memory or a volatile memory, and examples thereof include a dynamic random access memory (DRAM), a static random access memory (SRAM), or an electrically erasable programmable read-only memory (EEPROM).

The light source 101 may generate light by switching laser beams of a plurality of wavelengths. In this case, the calculating unit 110 may use electrical signals which are the reception results obtained by the element 106, of the acoustic waves propagating when radiating light of at least one wavelength among a plurality of wavelengths as an irradiation beam used for calculation of the optimal acoustic velocity. In this case, the light source 101 may select a wavelength in which the intensity of the electrical signal becomes strongest (that is, a wavelength in which the optical absorption coefficient of the light absorber becomes strongest) when the optimal acoustic velocity is calculated. However, the present invention is not limited thereto, but when the light absorber in the object 104 is an oxygenated hemoglobin and a reduced hemoglobin, a wavelength in which the absorption coefficients of the oxygenated hemoglobin and the reduced hemoglobin are substantially the same may be selected. By doing so, it is possible to calculate the optimal acoustic velocity with high accuracy and to calculate the delay period with high accuracy without any shift in the optical absorption amount of the irradiation beam in one of the two types of hemoglobin. By doing so, when a delay period table is calculated using light of a wavelength in which the absorption coefficient of the oxygenated hemoglobin is high and the absorption coefficient of the reduced hemoglobin is low, it is possible to prevent a decrease in the brightness of a reconstructed image of an area of interest in which many reduced hemoglobin are present. Thus, it is possible to prevent deterioration of the accuracy of the optimal acoustic velocity calculated from the brightness. However, the present invention is not limited thereto, but the calculating unit 110 may acquire electrical signals using light of a plurality of wavelengths actively when calculating the optimal acoustic velocity and calculate a total hemoglobin amount by performing image reconstruction using the electrical signals obtained by radiating light of the plurality of wavelengths. The calculating unit 110 may calculate the characteristics from an image indicating the total hemoglobin amount to calculate the optimal acoustic velocity and calculate the delay period from the optimal acoustic velocity.

The reconstructing unit 112 reconstructs the image of the object 104 using the electrical signals obtained by irradiating the object 104 with light and the delay period table recorded in the recording unit 111. In this image reconstruction, light is radiated to each area of interest to acquire electrical signals, and images are reconstructed in each area of interest to acquire the final characteristics information on the entire object. When images are reconstructed in each area of interest, the acoustic velocity value of each area of interest is read from the recording unit 111 and used. In the present example, universal back-projection used in the tomography technique is employed as an image reconstruction method which uses the data input to the recording unit 111. However, the present invention is not limited thereto, but a Fourier-domain back-projection, a synthetic aperture method, a time-reversal method, or the like may be used.

The reconstructing unit 112 performs the function of acquiring object information of the apparatus 100 and is positioned on the subsequent stage side within the processing of the apparatus 100. The driving unit 108b moves the receiving unit 105 to a reception position provided in each area of interest. The irradiating unit 102 radiates light around an area of interest immediately above the reception position. All elements 106 receive acoustic waves generated by radiating light to the reception position and output electrical signals to the processing unit 107.

The processing unit 107 converts the analog electrical signals output by the elements 106 into digital signals and records the digital signals in the signal memory in association with the elements 106 in a one-to-one correspondence. The reconstructing unit 112 reads the delay period of the area of interest corresponding to the reception position from the recording unit 111 and reconstructs the image of the area of interest using the delay period and the digital signal (reception data) recorded in the signal memory. This series of processes are performed on each area of interest. The reconstructing unit 112 combines all reconstructed images of the respective areas of interest to form the image of the entire measurement area to acquire the final characteristics information on the object in the entire measurement area. In this example, it is assumed that the characteristics information on the object may be the image information obtained by reconstructing the image of one area of interest and the image information obtained by combining the reconstructed images of all areas of interest.

Arithmetic units such as the selecting unit 109, the calculating unit 110, the reconstructing unit 112, and the like can be configured as a processor such as a CPU or a graphics processing unit (GPU) and an arithmetic circuit such as a field programmable gate array (FPGA) chip. These units may be formed of one processor or one arithmetic circuit and may be formed of a plurality of processors or arithmetic circuits.

The display unit 113 displays the images reconstructed by the reconstructing unit 112. A maximum intensity projection (MIP) image or a slice image may be displayed, and other display methods may be used. For example, a 3D image may be displayed from a plurality of different directions. Moreover, an inclination and a display region of a display image, a window level, and a window width may be changed by a user while checking the image displayed on the display unit 113. The display unit 113 may display the digital signal data obtained by converting the analog electrical signals output from the elements 106, stored in the delay period table or the signal memory. Moreover, these items of data may be displayed on an as-needed basis. Moreover, the reconstructed images based on irradiation of laser beams of different wavelengths may be displayed simultaneously. Further, the data may be displayed in a superimposed or synchronized manner, for example. Data that can be obtained by comparing reconstructed images based on different wavelengths, such as an oxygen saturation, a total hemoglobin amount, a glucose amount, and a molecule probe may be displayed in a parallel, superimposed, or synchronized manner, for example.

A position information acquiring unit 114 is a unit that acquires the position information of the receiving unit 106. The position information acquiring unit 114 can acquire position information when the receiving unit 106 receives acoustic waves. Light propagates through an object at a remarkably faster velocity than acoustic waves. Thus, the position information acquiring unit 114 may acquire the position information of the receiving unit 106 using a light generation timing as the acoustic wave reception timing. That is, the position information acquiring unit 114 may acquire the position information of the receiving unit 106 using light irradiation as a trigger. For example, a branch unit that branches light generated from the light source 101 may be provided, the branched light may be detected by an optical detector, and the position information acquiring unit 114 may acquire the position information of the receiving unit 106 using the output of the optical detector as a trigger. The position information acquiring unit 114 may be a position sensor, a laser sensor, a magnetic sensor, a linear encoder, a rotary encoder, or the like provided in the driving unit 108b.

### Reconfirmation of Present Example

In the present example, 512 elements 106 were arranged in a spiral form in the receiving unit 105. The light source 101 was configured to irradiate an object at a frequency of 20 MHz with a pulsed light beam having a wavelength of approximately 800 nm and a pulse width of 10 nm. The drive control unit 108a outputs a control signal so that the irradiating unit 102 radiates the pulsed light beam at a predetermined reception position (light irradiation position) and the element 106 can acquire signals in synchronism with the light irradiation. That is, the driving unit 108b outputs a control signal for moving the receiving unit 105 to a predetermined reception position to the driving unit 108b. The drive control unit 108a inputs a timing signal for radiating pulsed light beams at the reception position to the light source 101. The light source 101 generates the pulsed light beams at the above-described timing based on the timing signal.

The drive control unit 108a inputs a control signal for allowing the driving unit 108b to perform the following driving to the driving unit 108b. That is, the driving unit 108b moves the receiving unit 105 so as to perform light irradiation and receive photoacoustic waves while moving in a spiral form as described below about the center (the position of the irradiating unit 102) of the receiving unit 105 based on the control signal. The driving unit 108b sequentially moves the receiving unit 105 to the respective 2048 reception positions during the spiral scanning. Each element 106 receives acoustic waves at each reception position. That is, the receiving unit 105 receives acoustic waves in one reception position using the 512 elements 106. The element 106 receives the photoacoustic waves from the object 104 at 1048576 (2048×512) element positions in total and outputs analog electrical signals. The signal processing unit 107 converts the output analog electrical signals into digital signals, amplifies the digital signals using an amplifier, and records the amplified digital signals in the signal memory as reception data. The signal memory may simultaneously record the element positions of the elements 106 that have output the reception signals which are the base of the reception data in association with the reception data.

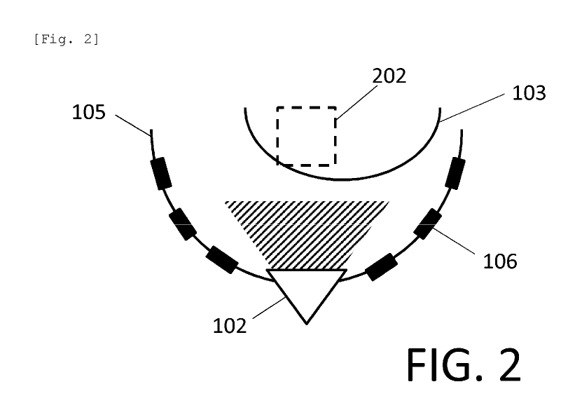

FIG. 2 is a schematic diagram illustrating the relation between an area of interest and a receiving unit of Example 1. In FIG. 2, although only one area of interest is illustrated for the sake of convenience, actually, a plurality of areas of interest are set so as to cover the entire surface of the measurement area. In FIG. 2, the selecting unit 109 sets a certain area of interest 202. Moreover, FIG. 2 illustrates a state in which the receiving unit 105 is moved to a reception position immediately below the area of interest 202 so that light can be radiated to the area of interest 202. That is, first, the selecting unit determines the area of interest 202, and then, the drive control unit 108a sets a reception position (light irradiation position) immediately below the area of interest 202 and outputs signals to the driving unit 108b so that the receiving unit is moved to the reception position.

In FIG. 2, the selecting unit 109 sets the area of interest 202 in a rectangular solid form to a range of 40 mm, 40 mm, and 40 mm in the x, y, and z directions of the object 104. The irradiating unit 102 is positioned immediately below the area of interest 202 and radiates light at that position. That is, this position is the reception position (light irradiation position) of the receiving unit 105. In FIG. 2, the position of the area of interest 202 is positioned at the curvature center of the receiving unit 105. All elements 106 receives acoustic waves generated from the area of interest 202 by radiating the light and outputs electrical signals to the subsequent stage.

The calculating unit 110 was configured to calculate the optimal acoustic velocity as a preceding stage for acquiring the delay period by assuming that in the positional relation of FIG. 2, the acoustic velocities in respective areas (water, the object, or the like) on the straight paths from the area of interest 202 to all elements 106 are equivalent. The calculating unit 110 used the golden section method as the optimal acoustic velocity calculation method. That is, the calculating unit 110 prepared various assumptive acoustic velocity values between 1450 m/s and 1650 m/s using the golden section method and reconstructed the images of the area of interest 202 at the respective assumptive acoustic velocities. That is, a number of reconstructed images for calculating the optimal acoustic velocity, corresponding to the number of assumptive acoustic velocities are formed.

In the receiving unit 105, water is filled between the receiving unit 105 and the acoustic matching material 103 so that the receiving unit 105 and the acoustic matching material 103 are acoustically coupled. The calculating unit 110 calculated the acoustic velocity in the water from the water temperature and set the optimal acoustic velocity in the water to 1486.9 m/s. The calculating unit 110 reconstructed images using the optimal acoustic velocity in the water and the assumptive acoustic velocity in the object 104 to calculate the optimal acoustic velocity in the area of interest 202. These processes were performed for all areas of interest to calculate the optimal acoustic velocity in all areas of interest.

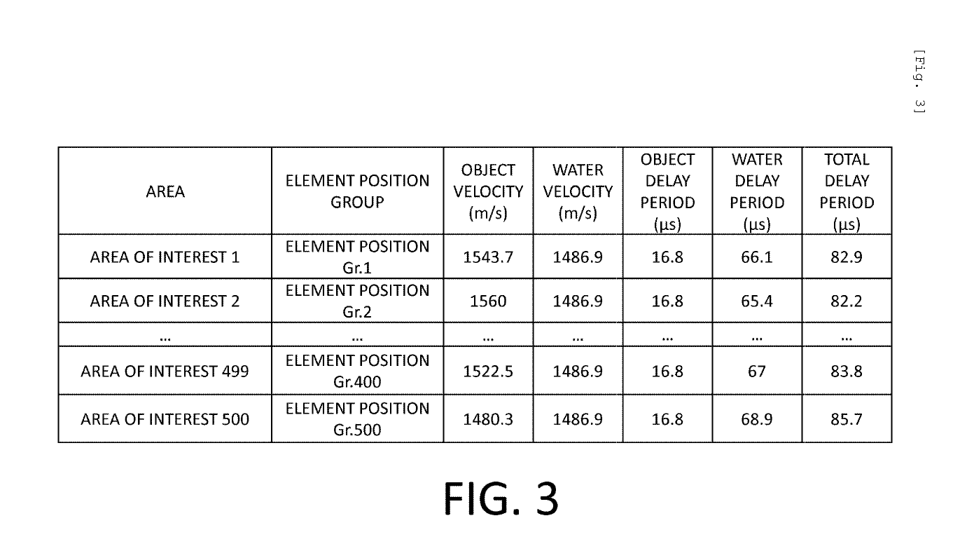

FIG. 3 is a diagram illustrating the result of a delay period table in Example 1. The recording unit 111 stores the following table. That is, the table stores an area of interest, an element position group when acoustic waves propagating from the area of interest are received, and the delay period in the object 104 and the delay period in the water calculated based on the acoustic velocity in the object 104 and the acoustic velocity in the water in association. The acoustic wave is the acoustic wave generated when the irradiating unit 102 radiated light when the unit is positioned immediately below the area of interest. The recording unit 111 records the central position of the area of interest and the size of the area of interest. Further, the recording unit 111 stores the acoustic velocity in the object, the acoustic velocity in the water, and a total delay period of the delay period in the object and the delay period in the water in association with each area of interest. That is, the recording unit 111 may store at least the acoustic velocity when the reconstructing unit 112 is configured to reconstruct images based on the acoustic velocity stored in the recording unit 111. Similarly, the recording unit 111 may store at least the delay period when the reconstructing unit 112 is configured to reconstruct images based on the delay period. Further, when the recording unit 111 stores the total delay period rather than storing the delay period in the water and the delay period in the object, it is possible to save the memory capacity as compared to when both the delay period in the water and the delay period in the object are stored.

The selecting unit 109 sets a plurality of areas of interest within the range of the measurement area of 128 mm×128 mm×64 mm. The reconstructing unit 112 reads the optimal reception data in an image reconstruction target area of interest from the signal memory and performs image reconstruction in each area of interest. The reconstructing unit 112 combines the reconstructed images of the respective areas of interest to acquire a final reconstructed image of the entire measurement area including the object 104. When an area of interest does not fall within the measurement area, the image reconstruction may be performed using the average value of the optimal acoustic velocities of the neighboring areas of interest.

### Example 2

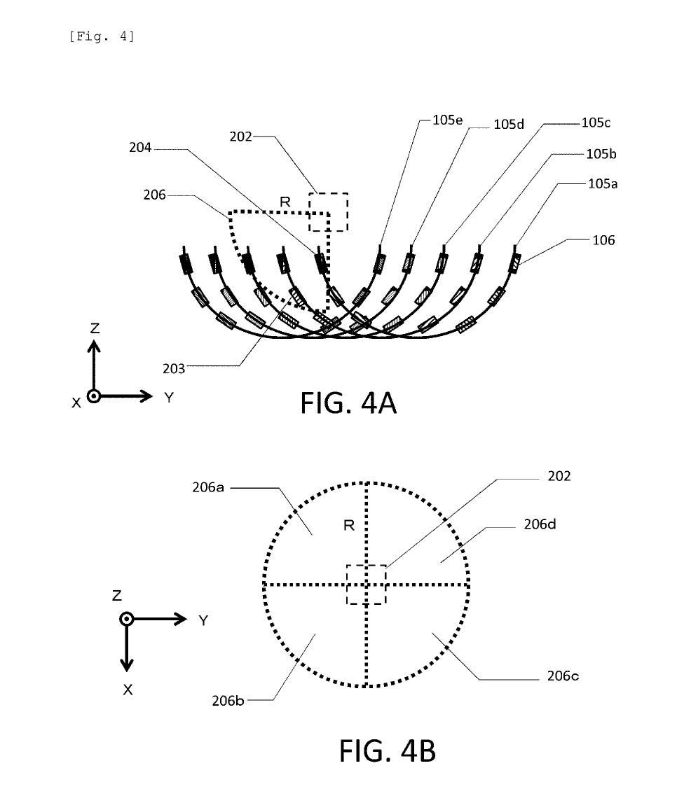

FIGS. 4A and 4B are schematic diagrams illustrating Example 2 of the object information acquiring apparatus of the present invention. The same constituent elements as those of Example 1 will be denoted by the same reference numerals, and the description thereof will be omitted unless it is necessary. FIG. 4A is a diagram illustrating element positions at five reception positions and an area of interest 202. FIG. 4B is a schematic diagram illustrating the area of interest 202 and four selection areas 206 set thereto. Selection areas 206a, 206b, 206c, and 206d are set as indicated by broken lines having a shape obtained by dividing a hemispherical shape around the area of interest 202 into four equal parts. Some of the element positions illustrated in FIG. 4A are included in any one of the selection areas 206a, 206b, 206c, and 206d. The calculating unit 110 calculates the optimal acoustic velocity using the four element position groups set in this manner as the preceding stage for calculating the delay period.

Hereinafter, the difference between the present example and Example 1 will be described. The selecting unit 109 divides the measurement area into 400 parts (10×10×4) and sets areas of interest each having the size of 12.8 mm×12.8 mm×16 mm so as to cover the entire measurement area. However, the present invention is not limited thereto, but the areas of interest may be set in an area only in which the acoustic velocity in the measurement area is to be calculated. As illustrated in FIGS. 4A and 4B, acoustic wave detection elements 203 (in FIG. 4A, four elements) included in four broken-line areas which are located close to the receiving unit 105 within a radius R from the center of the area of interest 202 and of which the latitude and longitude are π/2 are selected. Here, “select” means that the electrical signals which are the reception results that the four acoustic wave detection elements 203 received at the respective reception time are used in image reconstruction. In this example, only the elements 106 positioned on the ZY plane are illustrated for the sake of convenience. However, those acoustic wave detection elements which are arranged three-dimensionally in the XYZ directions and which are included in the selection areas 206a to 206d are also actually included in the selected acoustic wave detection elements. Further, the reception sensitivity of the acoustic wave detection elements included in the selection area 206 may decrease in the following cases. That is, the angle between the direction from the reception surface of the element 106 to the area of interest 202 in which acoustic waves are generated and the direction (the direction in which the reception sensitivity is highest) normal to the reception surface is too large, and the element 106 may be unable to receive high-frequency components of the acoustic waves from the area of interest due to its directivity. The accuracy of the electrical signals obtained when such an acoustic wave detection element 203 received acoustic waves from the area of interest 202 is low. Thus, such electrical signals are not used when reconstructing the image of the area of interest 202.

Specifically, for example, the angle between the direction in which the reception sensitivity of the acoustic wave detection element 204 is highest and the direction from the acoustic wave detection element 204 to the area of interest 202 is 40° or more. This angle is equal to or larger than the half-value angle of the acoustic wave detection element 204. That is, the element 204 cannot receive the acoustic waves propagating from the area of interest 202 with sufficient reception sensitivity. On the other hand, the angle of the direction in which the reception sensitivity of the acoustic wave detection element 203 is highest and the direction from the acoustic wave detection element 203 to the area of interest 202 is smaller than 40°, which is smaller than the half-value angle of the acoustic wave detection element 203. That is, the element 203 can receive the acoustic waves propagating from the area of interest 202 with sufficient reception sensitivity. Thus, the acoustic wave detection element 204 is excluded from the acoustic wave detection elements to be selected. The selecting unit 109 selects the reception data received at an element position used for calculation of the optimal acoustic velocity in each of the areas of interest of which the positions are fixed. Although it is not always necessary to exclude the acoustic wave detection element 204, if high accuracy of the reception sensitivity is not required, the electrical signals output from the acoustic wave detection element 204 may be used for image reconstruction of the area of interest 202. Further, although the selection criterion is set to the half-value angle of 40°, the criterion is not limited thereto, but an angle which can be sufficiently used for desired image reconstruction may be used as a threshold and other criteria other than the angle may be used.

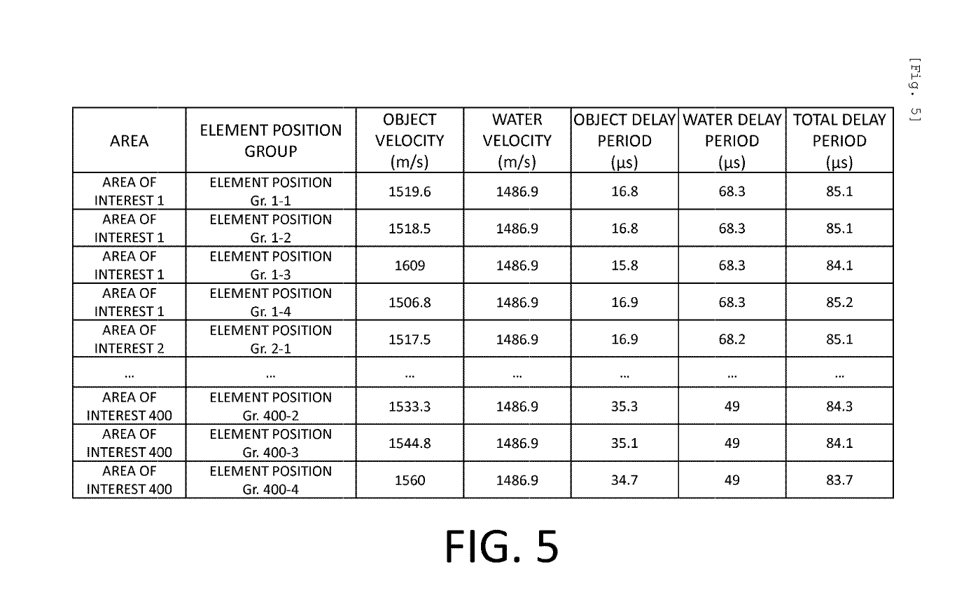

FIG. 5 is a diagram illustrating a delay period table of Example 2. This table is recorded in the recording unit 111. The calculating unit 110 sets an object assumptive acoustic velocity and a water assumptive acoustic velocity for calculation of the optimal acoustic velocity in each of the four acoustic wave detection element groups as a preceding stage for calculating the delay period. Moreover, the object optimal acoustic velocity and the water optimal acoustic velocity used when imaging the area of interest 202 based on the assumptive acoustic velocities are calculated for each of the four groups. The calculating unit 110 calculates the delay period for each of the four groups from the calculation result and records the delay period in the recording unit 111. That is, the acoustic wave detection element group and the object delay period and the water delay period calculated by the acoustic wave detection element group are recorded in the recording unit 111 in association with the area of interest 202. This process is performed for all 400 areas of interest, and such a delay period table as illustrated in FIG. 5 is stored in the recording unit 111. After the above-described processes are performed, photoacoustic measurement is performed on the object again. The reconstructing unit 112 performs image reconstruction using the digital data which is the reception result and the delay period table recorded in the above process and displays the reconstructed image on the display unit 113 and the delay period table on the display unit 113. Similarly to FIG. 3, the recording unit 111 stores the acoustic velocity in the object, the acoustic velocity in the water, and a total delay period of the delay period in the object and the delay period in the water in association with each area of interest. That is, the recording unit 111 may store at least the acoustic velocity when the reconstructing unit 112 is configured to reconstruct images based on the acoustic velocity stored in the recording unit 111. Similarly, the recording unit 111 may store at least the delay period when the reconstructing unit 112 is configured to reconstruct images based on the delay period. Further, when the recording unit 111 stores the total delay period rather than storing the delay period in the water and the delay period in the object, it is possible to save the memory capacity as compared to when both the delay period in the water and the delay period in the object are stored.

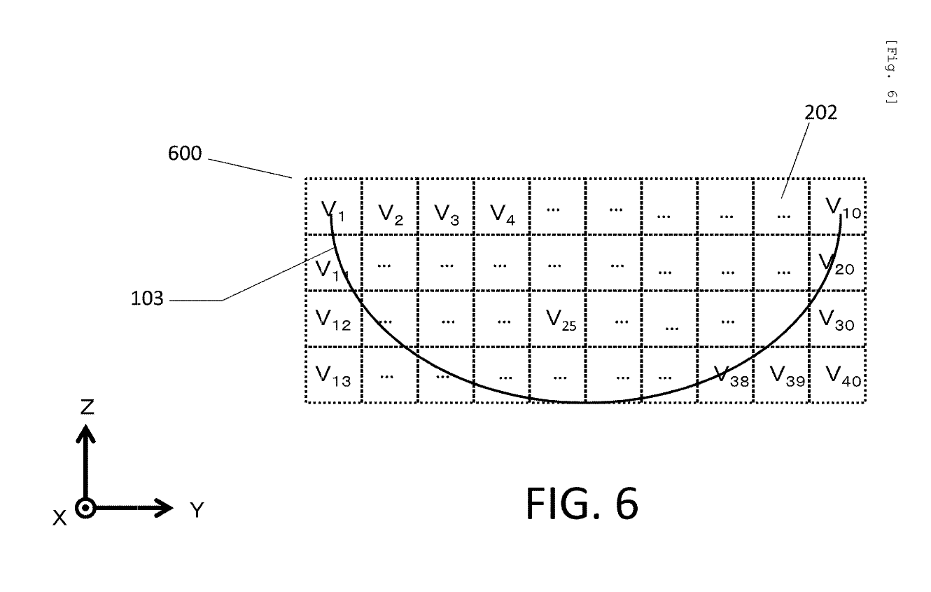

FIG. 6 is a schematic diagram illustrating the optimal acoustic velocity in each area of interest of Example 2. FIG. 6 is a diagram illustrating 400 areas of interest obtained by dividing a measurement area 600 and the optimal acoustic velocity values Vi to V40 corresponding to the respective areas of interest in a certain ZY plane in a superimposed manner. In the present example, although the acoustic velocity values are displayed in numerical values as in FIG. 6, the acoustic velocity may be displayed in colors. For example, when a certain acoustic velocity has a value of 1450 m/s, the acoustic velocity may be displayed in blue. When a certain acoustic velocity has a value of 1650 m/s, the acoustic velocity may be displayed in red. Moreover, acoustic velocities between the two acoustic velocities may be displayed with gradations. In the present example, as compared to Example 1, a group of two or more elements 106 in which the propagation paths of photoacoustic waves from the area of interest can be considered to be the same is selected, and the optimal acoustic velocities of the areas of interest are calculated using the acoustic wave detection element group. Thus, the edge sharpness of the image is improved when the entire measurement area is imaged.

### Example 3

The light radiated to the object 104 in Examples 1 and had a wavelength of 800 nm. However, in the present example, a pulsed light beam having a wavelength of 756 nm is also radiated to the object 104. Here, the difference from Examples 1 and 2 will be described. After the measurement in Example 1 was performed, the laser wavelength was switched to 756 nm, the light is radiated to the object 104, and the digital data (acquisition signals) obtained by receiving photoacoustic waves based on the irradiation was recorded in a signal memory (not illustrated). Examples of light absorbers which can better absorb light of these wavelengths 800 nm and 756 nm when such measurement was performed using the wavelengths include reduced hemoglobin and oxygenated hemoglobin in the object 104. Light having the wavelength of 800 nm in which two types of hemoglobin have approximately the same absorption coefficient was radiated and a delay period table was calculated using the digital data obtained by receiving photoacoustic waves based on the irradiation. The delay period table is calculated in the same manner as Example 2. Subsequently, an operator input the delay period table for the wavelength 800 nm and the digital data acquired by radiating the light of the wavelength 756 nm to the reconstructing unit 112, and the reconstructed image based on irradiation of the light of the wavelength 756 nm was acquired. Moreover, the delay period table for the wavelength 800 nm and the digital data obtained by radiating light of the wavelength 800 nm were input to the reconstructing unit 112 and the image based on the wavelength 800 nm was reconstructed. Two reconstructed images obtained by radiating the light of the wavelengths 756 nm and 800 nm were displayed on the display unit 113 in parallel. By creating the delay period table using the wavelength 800 nm and applying the delay period table to the measurement based on radiation of the light of an additional wavelength, it is possible to reduce the time to calculate the delay period table for the other wavelength.

### Example 4

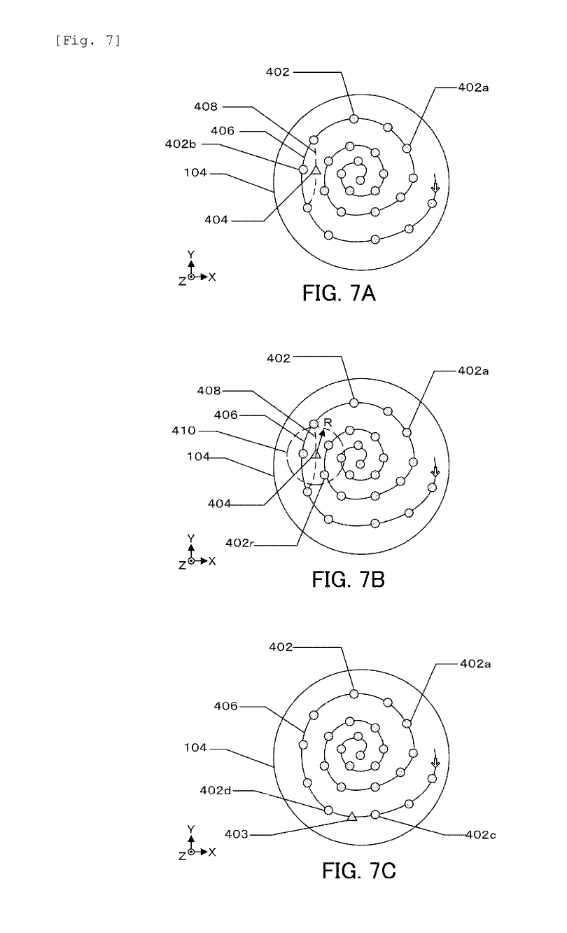

FIGS. 7A to 7C are schematic diagrams illustrating a scanning method according to Example 4 of the object information acquiring apparatus of the present invention. The same constituent elements as those of Example 1 will be denoted by the same reference numerals and the description thereof will be omitted. FIGS. 7A to 7C are schematic diagrams when a scanning trajectory on the ZX plane of the receiving unit 105 is seen from the Z-axis direction and illustrates that the receiving unit 105 scans in a spiral form from the outside of the object 104 toward the inner center of the object 104. In FIGS. 7A to 7C, hollow arrows indicate the scanning directions, and the receiving unit 105 scans from the outside toward the inner center along the spiral form. The receiving unit 105 is scanned by the drive control unit 108a and the driving unit 108b so that the position (projection position) on the ZX plane of the curvature center of the receiving unit 105 projected on the ZX plane follows the spiral trajectories illustrated in FIGS. 7A to 7C. Moreover, in the present example, although the areas of interest and the reception positions correspond in one-to-one correspondence, the present invention is not limited thereto, but a plurality of reception positions may correspond to one area of interest as in Example 2, for example. When the projection position is identical to reception positions 402 arranged along the spiral line, light is radiated to the object and photoacoustic waves are received. For example, light is radiated to the object when the projection position is positioned at a reception position 402a. The propagating photoacoustic waves are received by a plurality of elements 106. The digital data is acquired from the electrical signals output as the reception results and is stored in the signal memory. This storing process is performed for all reception positions.

Subsequently, the image of the entire measurement area is formed based on the data stored in the signal memory and the delay period which is the data recorded in the recording unit 111. The image is formed according to the following method, for example. That is, the images of the respective areas of interest are sequentially reconstructed to form the image of all areas of interest, whereby the image of the entire range of the reconstructed image is formed.

Specifically, the delay period in the area of interest corresponding to the reception position 402a, for example, is read from the recording unit 111. The delay period is calculated by measuring the object 104 or other objects in advance and is stored in the recording unit 111. The image of the area of interest corresponding to the reception position 402a is reconstructed based on the read delay period and the digital data corresponding to the reception position 402a stored in the signal memory. After this process is performed for all areas of interest, as described above, the reconstructed images of all areas of interest are combined to form and acquire the image of the entire measurement area. The delay periods corresponding to the respective areas of interest are stored in the recording unit 111.

However, due to malfunction or the like of the drive control unit 108a or the driving unit 108b, the projection position may be positioned at a position 404 shifted from the spiral lines in FIGS. 7A to 7C, which are predetermined trajectories. That is, when the projection position is shifted to the position 404 from the predetermined reception position 402b, the scanning trajectory follows a trajectory 408. Light is radiated to the object 104 at the position 404, and photoacoustic waves are received for the area of interest in the object 104 at a position shifted to the position 404 from the predetermined reception position 402b. However, in some cases, the delay period corresponding to the positional relation between the area of interest and the position 404 is not stored in the recording unit 111. Thus, the reconstructing unit 112 determines the delay period corresponding to the area of interest and the position 404 using the information on the delay period stored in the recording unit 111.

A first method uses the so-called nearest neighbor method. Specifically, in FIG. 7A, the delay period in the area of interest corresponding to a predetermined reception position 402b nearest from the shifted position 404 is read from the recording unit 111. Moreover, the image of the shifted area is reconstructed based on the delay period and the electrical signal.

A second method uses linear interpolation. That is, the acoustic velocities or delay periods corresponding to the reception positions 402r positioned within a predetermined radius R from the position 404 are weighted according to the distance from the reception positions 402r to the position 404 and the weighted values are added. That is, the larger the distance, the lighter the weight applied to the acoustic velocity or the like, whereas the smaller the distance, the heavier the weight applied to the acoustic velocity or the like. Specifically, for example, in FIG. 7B, four reception positions 402r are present within the range (corresponding to the distance) of the radius R. The distances of the reception positions 402r from the position 404 are defined as r1, r2, r3, and r4 in descending order, and the acoustic velocities corresponding to the reception positions 402r at the positions of the radii r1, r2, r3, and r4 are defined as v1, v2, v3, and v4. In this case, the weight w at the reception position is set to (1/r)/(1/r1+1/r2+1/r3+1/r4). When the weights at the respective reception positions are defined as w1, w2, w3, and w4, the acoustic velocity vo used for image reconstruction based on the reception at the position 404 can be calculated as 1/vo=1/{w1/v1+w2/v2+w3/v3+w4/v4}.

In this way, even when the acoustic velocity vo (corresponding to the acquisition result) used for image reconstruction based on the reception at the position 404 is calculated and photoacoustic waves are received at the position 404, it is possible to use the electrical signals output based on the reception without wasting the electrical signals. Further, it is possible to prevent the electrical signals obtained based on the reception at the shifted position 404 from decreasing the accuracy of the image reconstruction. Various other interpolation methods may be used without being limited to the above-described interpolation method. Moreover, in this example, although the acoustic velocity vo is calculated by interpolation and is used for image reconstruction, the present invention is not limited thereto, but the above-described interpolation may be applied to the delay period and the delay period calculated by interpolation may be used for image reconstruction.

Moreover, although the position 404 shifted from the spiral trajectory has been described, as illustrated in FIG. 7C, photoacoustic waves may be received at a position 403 different from the predetermined reception position 402 even if the position 403 is not shifted from the spiral trajectory. That is, the position 403 is on a spiral trajectory extending from a reception position 402c to a reception position 402d. In this case, an acoustic velocity v403 in the element 106 in a shifted area corresponding to the shifted position 403 may be calculated by interpolation using the acoustic velocities vc and vd recorded in the recording unit 111 corresponding to the predetermined reception positions 402c and 402d, for example. Here, the “shifted area” is an area of which the position is shifted from the area of interest and which includes an area in which the direction in which the reception sensitivity of the element 106 at the position 403 is highest is focused. The acoustic velocity is calculated by the following method, for example. That is, the reconstructing unit 112 calculates a radius Rc to the position 403 from the position 402c which is one of the reception positions having the position 403 interposed and a radius Rd to the position 403 from the position 402d which is the other of the reception positions having the position 403 interposed. After that, the acoustic velocity v403 is calculated by linear interpolation expressed by v403=vc×rd+vd×rc. That is, the larger the distance from the position 403, the lighter the weight applied to the acoustic velocity at the reception position corresponding to the distance, whereas the smaller the distance, the heavier the weight applied to the acoustic velocity. After that, the reconstructing unit 112 reconstructs the image of the shifted area including an acoustic wave generation source in which acoustic waves are generated by irradiation of light at the position 403 using the calculated acoustic velocity v403 and the electrical signal output based on the reception at the position 403. However, the present invention is not limited thereto, but the interpolation method used in the states of FIGS. 7A and 7B may be applied to the state of FIG. 7C, and other various interpolation methods may be used.

With the above, interpolation can be applied to a case in which an area of interest is set, photoacoustic waves are received at a position shifted from the predetermined reception position 402 corresponding to the area, and information on the acoustic velocity and the delay period corresponding to the shifted position is not recorded in the recording unit 111. That is, interpolation can be performed using the data of the acoustic velocities or the delay periods corresponding to the areas of interest corresponding to the reception positions recorded in the recording unit 111. As a result, it is possible to reduce the time and the cost taken to acquire the object information and to acquire high-accuracy object information.

### Example 5

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

### Other Examples

A person having ordinary skill in the art could easily conceive a new system by appropriately combining various techniques of the respective examples. Thus, the system obtained by such combinations fall within the scope of the present invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-226951, filed on Nov. 7, 2014, which is hereby incorporated by reference herein in its entirety.

### REFERENCE SIGNS LIST

104: OBJECT
106: ELEMENT
107: SIGNAL PROCESSING UNIT
108A: DRIVING CONTROL UNIT
108B: DRIVING UNIT
109: SELECTING UNIT
111: RECORDING UNIT
112: RECONSTRUCTING UNIT

1.-14. (canceled)

15. An object information acquiring apparatus for acquiring characteristic information at a position of interest in an object by using electrical signals obtained by receiving an acoustic wave generated from the object by a receiving unit, the object information acquiring apparatus comprising:
a recording unit in which information on the time taken for the acoustic waves generated from the position of interest to reach the receiving unit and information on each of a plurality of predetermined reception positions are stored in association with each other, with respect to each of the plurality of predetermined reception positions; and
a time information acquiring unit configured to calculate, when the receiving unit receives the acoustic waves at a position shifted from the predetermined reception position, information on the time taken for the acoustic waves generated from the position of interest to reach the shifted position using the information on the time associated with the plurality of the predetermined reception positions and stored in the recording unit,
a characteristic information acquiring unit configured to acquire the characteristics information on the position of interest, using the electrical signals and the information on the time taken for the acoustic waves generated from the position of interest to reach the shifted position.

16. The object information acquiring apparatus according to claim 15, wherein the receiving unit includes a plurality of acoustic wave detection elements configured to receive the acoustic waves propagating through the object and to output electrical signals.

17. The object information acquiring apparatus according to claim 16, wherein the information on the time is information obtained in advance by dividing a distance from the position of interest to the acoustic wave detection element at the predetermined reception position by an acoustic velocity corresponding to a path from the position of interest to the acoustic wave detection element at the predetermined reception position.

18. The object information acquiring apparatus according to claim 16, wherein
when the plurality of acoustic wave detection elements receive acoustic waves propagating from a position shifted from the position of interest of the object at a position shifted from the predetermined reception position and output electrical signals, and
the characteristics information acquiring unit acquires the characteristics information on the object based on the electrical signals and the information on the time associated with the predetermined reception position at a position nearest to the shifted position.

19. The object information acquiring apparatus according to claim 15, further comprising:
the receiving unit; and
a position control unit configured to move the receiving unit to the plurality of predetermined reception positions.

20. The object information acquiring apparatus according to claim 19, wherein
the position control unit moves the receiving unit along a predetermined moving path,
when the plurality of acoustic wave detection elements receive acoustic waves propagating from the position of interest of the object at a position shifted from the predetermined reception position on the predetermined moving path and output electrical signals,
the time information acquiring unit performs interpolation based on the information on the time associated with first and second predetermined reception positions to acquire information on the time taken for the acoustic waves generated at the position of interest to reach the acoustic wave detection element at the shifted position,
the characteristic information acquiring unit acquires the characteristics information on the object in the shifted position based on the acquired information and the output electrical signal, and
the shifted position is arranged between the first predetermined reception position and the second predetermined reception position.

21. The object information acquiring apparatus according to claim 20, wherein the time information acquiring unit performs the interpolation based on a ratio between a distance from the first predetermined reception position to the shifted position and a distance from the second predetermined reception position to the shifted position.

22. The object information acquiring apparatus according to claim 19, wherein the position control unit continuously moves the receiving unit.

23. The object information acquiring apparatus according to claim 19, wherein the position control unit moves the receiving unit to the predetermined reception position in a stepwise manner.

24. The object information acquiring apparatus according to claim 20, wherein the predetermined moving path is a spiral moving path.

25. The object information acquiring apparatus according to claim 15, further comprising:
an irradiating unit that irradiates the object with light to allow the acoustic waves to propagate.

26. The object information acquiring apparatus according to claim 25, wherein the predetermined reception position is a position at which the light can be radiated to the position of interest.

27. An object information acquiring method performed by an object information acquiring apparatus for acquiring characteristic information at a position of interest in an object by using electrical signals obtained by receiving an acoustic wave generated from the object by a receiving unit, the object information acquiring method comprising:
a step of acquiring information on the time taken for the acoustic waves generated from the position of interest to reach the receiving unit and information on each of a plurality of predetermined reception positions, with respect to each of the plurality of predetermined reception positions;
a time information acquiring step of calculating, when the receiving unit receives the acoustic waves at a position shifted from the predetermined reception position, information on the time taken for the acoustic waves generated from the position of interest to reach the shifted position using the information on the time associated with the plurality of the predetermined reception positions and stored in the recording unit; and
a characteristic information acquiring step of acquiring the characteristics information on the position of interest, using the electrical signals and the information on the time taken for the acoustic waves generated from the position of interest to reach the shifted position.

28. The object information acquiring method according to claim 27, wherein
the receiving unit includes a plurality of acoustic wave detection elements configured to receive the acoustic waves propagating through the object and to output electrical signals,
the plurality of acoustic wave detection elements is moved along a predetermined moving path,
when the plurality of acoustic wave detection elements receive acoustic waves propagating from the position of interest of the object at a position shifted from the predetermined reception position on the predetermined moving path and output electrical signals,
in the time information acquiring step, the information on the time taken for the acoustic waves generated at the position of interest to reach the acoustic wave detection element at the shifted position is acquired by performing an interpolation based on the information on the time associated with first and second predetermined reception positions,
in the characteristic information acquiring step, the characteristics information on the object in the shifted position is acquired based on the acquired information and the output electrical signal, and
the shifted position is arranged between the first predetermined reception position and the second predetermined reception position.

29. The object information acquiring method according to claim 28, wherein in the time acquiring step, the interpolation is performed based on a ratio between a distance from the first predetermined reception position to the shifted position and a distance from the second predetermined reception position to the shifted position.

30. A non-transitory computer readable storing medium recording a computer program for causing a computer to perform the object information acquiring method according to claim 27.
